(19) United States
(12) Patent Application Publication
(10) Pub. No.: US 2012/0072236 A1
(43) Pub. Date: Mar. 22, 2012

(54) INSULIN PEN DATA RECORDING AND TRANSMISSION DEVICE

(76) Inventor: Benjamin Atkin, North Miami Beach, FL (US)

(21) Appl. No.: 13/205,453

(22) Filed: Aug. 8, 2011

Related U.S. Application Data
(60) Provisional application No. 61/371,322, filed on Aug. 6, 2010.

(51) Int. Cl. G06Q 50/24; A61M 5/31

(52) U.S. Cl. 705/3; 604/189

(57) ABSTRACT

A system and method is provided whereby an electromechanical device is paired with an insulin delivery device such that said pairing results in the recordation of insulin delivery date, time and amount.

### INDEX TO RELATED APPLICATIONS

This Application claims the benefit of U.S. Provisional Patent Application No. 61/371,322 filed Aug. 6, 2011 the disclosure of which is incorporated herein by reference in its' entirety.

### BACKGROUND OF THE INVENTION

Insulin pens are devices that can provide an easy way to carry and give insulin (e.g., when away from home). The pens provide a convenient and accurate alternative to draw up and measure individual doses . There are two types of conventional insulin pens: those that are completely disposable after providing a single dose of insulin; and those that use disposable cartridges to provide multiple doses. An insulin pen typically has three components: a base which contains the mechanism for measuring the correct dose; a holder for the cartridge of insulin; and a cap, which is removed and replaced by the needle prior to the injection.

### SUMMARY OF THE INVENTION

The present invention relates to an existing insulin delivery device, such as an insulin pen. The present invention is an electromechanical device, which is affixed to the pen. When the user rotates the top of the pen to select the prescribed dosage of insulin to be injected into the body, the device mechanically measures the distance traveled by the plunger inside the pen from its original position of “zero” to the position where the plunger rests immediately prior to injection. The displacement of the plunger is converted to a measurement of units of insulin. When the plunger is depressed and returns to the “zero” position indicating an insulin injection, the amount of insulin injected is recorded by the device together with the time and date, and stored in the device's memory. Periodically the device of the present invention is connected to a data transmission device which downloads the data stored in the device's memory and transmits such data electronically via SMS, cellphone, Bluetooth, WiFi or other means to the users medical record.

The data transmitted regarding injection of the insulin includes (but is not limited to), for at least one injection, at least one dosage amount, the time of injection and the date of injection. The data transmitted may be coupled with other data collected by the user via electronic or other means and combined to provide a more complete record of blood glucose measurements, treatments and subsequent diabetes disease management. The following graph and chart are an example of the data collected from the device, merged with a user's blood glucose readings.

### BRIEF DESCRIPTION OF THE DRAWINGS

FIG. 1 is a perspective view showing the insulin pen of the present invention with an upload module.

FIG. 2 is a perspective view showing the insulin pen of the present invention.

FIG. 3 is a perspective view showing the insulin pen of the present invention.

FIG. 4 is an expanded partial perspective view showing the insulin pen of the present invention.

### DETAILED DESCRIPTION OF THE INVENTION

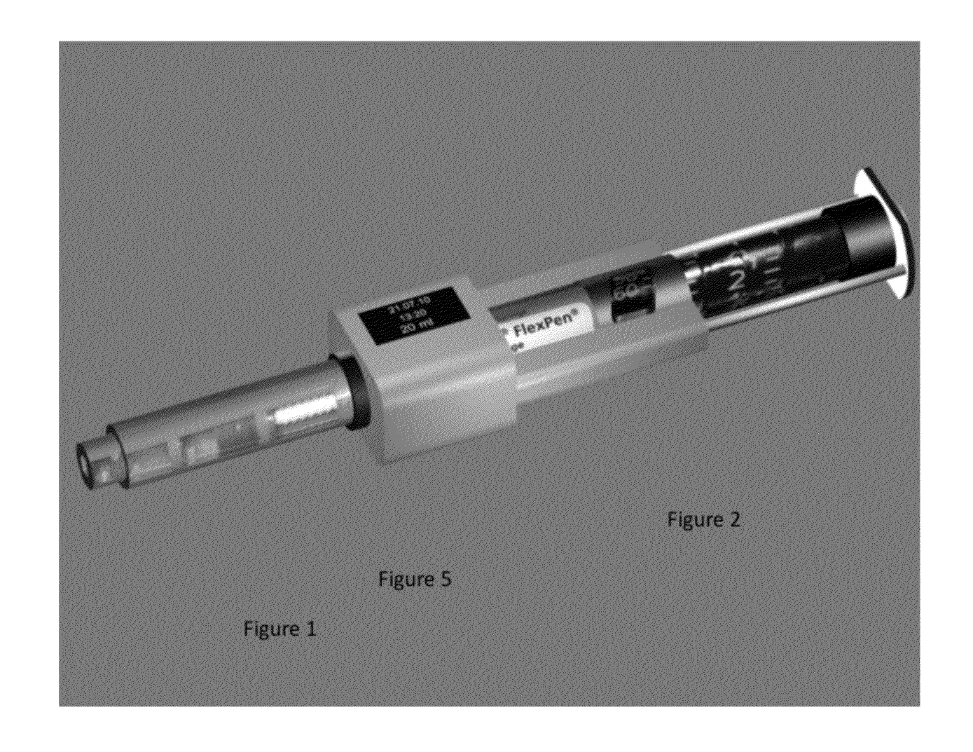

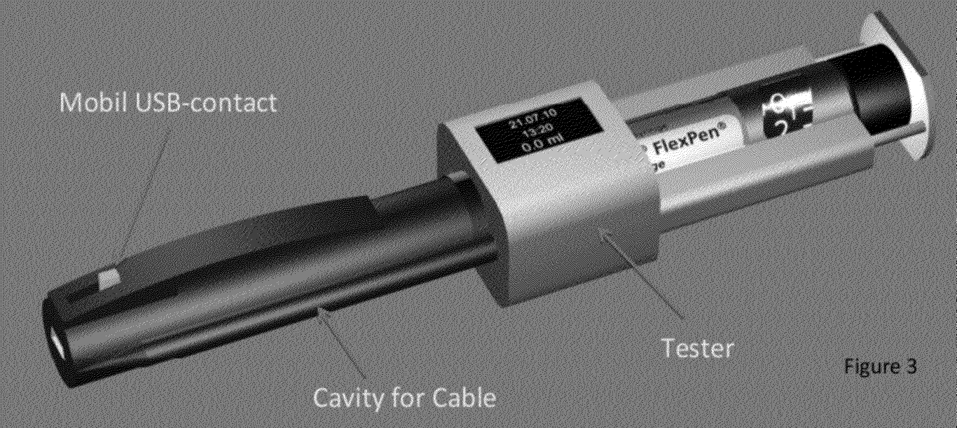

In the following illustration an insulin injection device is provided, comprising: a container which holds the insulin FIG. 1; and a data recorder and transmitter FIG. 2, which collects data regarding injection of insulin including (but not limited to), the dosage for the injection, the time of injection and the date of injection.

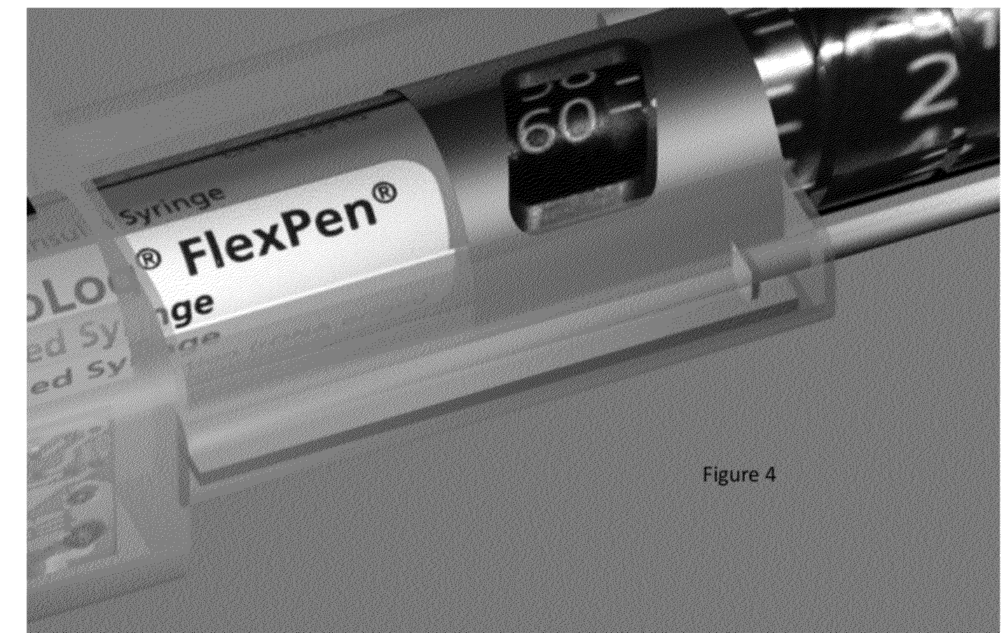

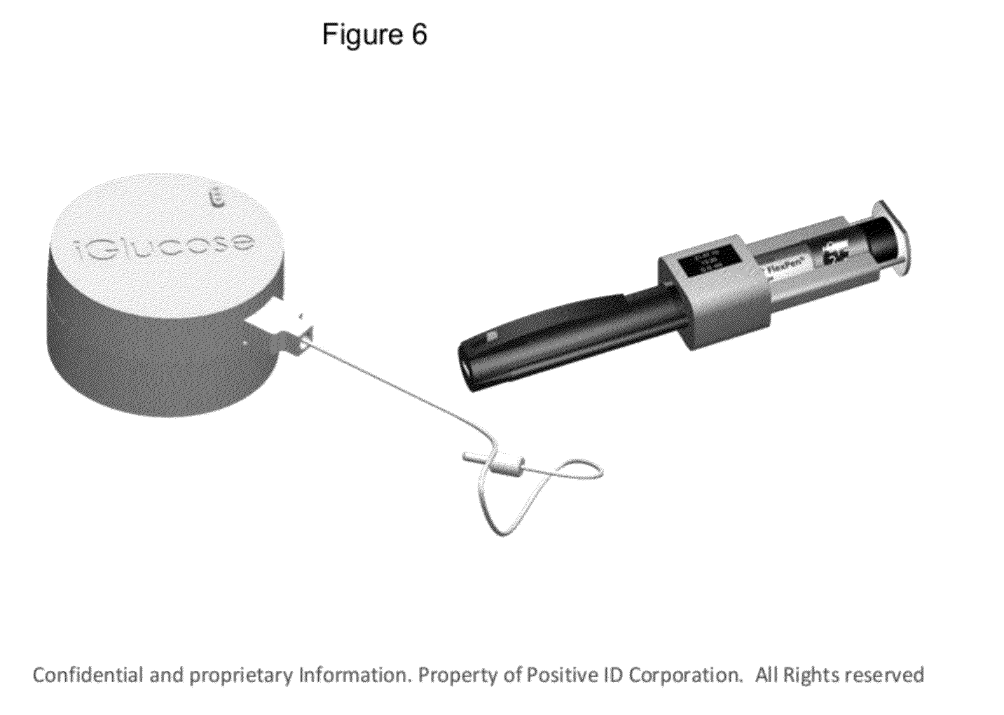

When the insulin dosage is selected by rotating the insulin doser FIG. 3, the slide bar FIG. 4 moves correspondingly with the plunger in FIG. 5. Once the plunger is depressed the microprocessor in the device records the dosage, time and date.

In FIG. 6, another illustration of the device, the device is attached to a data transmitter via cable or other means and the data transmitter, containing software extracts the data from the devices stored memory and transmits said data to a hosting site where the users medical records are maintained. Software then integrated the received data from the device to the users medical records.

The data regarding the injection of insulin may include, but not is not limited to, at least one dosage amount, the time of injection and the date of injection. At least some of the data may be stored in the memory at the time of each injection and at least some of the stored data may be transmitted by the data transmitter at a later time.

The data transmitter may be powered by a battery or the data transmitter may be powered by a photovoltaic cell or the data transmitter may be powered by a battery which may be charged by a photovoltaic cell.

The present invention easily couples with a conventional insulin pen and may be removed from one pen and coupled with another pen at the users discretion. The device is a recording and transmitting device so that dosages of insulin may be electronically recorded for an individual user. The invention does not require a user to manually or otherwise record data (such as measurements) taken from the pen. Instead, data from the device uses a means of communication—such as wireless technology—to the users electronic medical record (EMR). The EMR may consist of a central server apparatus that collects the information from the insulin pen. Transmission of the data by the present invention may occur with or without direct physical connection to the device. The transmission from the device may occur at predetermined times (e.g. downloads).

The EMR at which data is recorded, stored and/or analyzed, may be integrated with lifestyle data as well as extrinsically generated information on a patient's medical treatment.

While the invention has been described in its preferred form or embodiment with some degree of particularity, it is understood that this description has been given only by way of example and that numerous changes in the details of construction, fabrication, and use, including the combination and arrangement of parts, may be made without departing from the spirit and scope of the invention.

I claim:

1. A system for monitoring and recording insulin administration comprising:
an insulin delivery device;
an electromechanical device operatively associated with said insulin delivery device;
said electromechanical device constructed and arranged to selectively record any of time, date, amount of insulin delivered or combinations thereof.

2. The system of claim 1 wherein said insulin delivery device delivers injectable insulin to a user.

3. The system of claim 1 wherein said insulin delivery device is an insulin pen.

4. The system of claim 1 wherein said electromechanical device is constructed and arranged to be interchangeable with a plurality of insulin delivery devices.

5. The system of claim 1 wherein said electromechanical device has a physical memory for storing said record.

6. The system of claim 1 wherein said electromechanical device has a transmission mechanism.

7. The system of claim 1 wherein said electromechanical device has a transmission mechanism activated by a user.

8. The system of claim 1 wherein said electromechanical device has a transmission mechanism activated automatically at a set time.

9. The system of claim 1 wherein said electromechanical device has a transmission mechanism activated by a user configured to transmit to a receiving device.

10. A method of assembling an electronic medical record comprising:
providing an electromechanical device according to the system of claim 1;
transmitting data from said electromechanical device relating to any of time, date, amount of insulin delivered, or combinations thereof;
receiving said data in a receiving device;
sorting said data;
assembling an electronic medical record from said data;
outputting said electronic medical record based on said data.

11. The method of claim 10 wherein said electronic medical record is assembled from more than one transmission from said electromechanical device.
